(19) United States
(12) Patent Application Publication
(10) Pub. No.: US 2023/0256258 A1
(43) Pub. Date: Aug. 17, 2023

(54) FOOTWEAR AND INSERT HAVING THERAPEUTIC LIGHT SOURCE

(71) Applicant: Biothread LLC, Wayne, PA (US)

(72) Inventors: Jay Tapper, Wayne, PA (US); Daniel Bish, New York, NY (US); Daniel Shuter, New York, NY (US); Kristien Del Ferraro, Stamford, CT (US)

(21) Appl. No.: 17/836,030

(22) Filed: Jun. 9, 2022

Related U.S. Application Data
(63) Continuation-in-part of application No. 17/671,764, filed on Feb. 15, 2022.

(51) Int. Cl. A61N 5/06; A43B 7/00; A43B 17/00

(52) U.S. Cl. CPC A61N 5/06; A43B 7/00; A43B 17/00; A61N 2005/063; A61N 2005/0645; A61N 2005/0659; A61N 2005/0663

(57) ABSTRACT

An insole for an article of footwear includes an insole support member and a side-emitting optical fiber fixed with respect to the insole support member. The insole support member is made from a cushioning material and includes a top surface and a bottom surface. The side-emitting optical fiber is configured to project radiation having a therapeutic wavelength along the length of and outwardly through a side of the side-emitting optical fiber and toward a wearer’s foot when the insole is positioned in the article of footwear and the article of footwear is worn. At least a majority of an uppermost surface of the side-emitting optical fiber is disposed beneath a foot-contacting surface that is in contact with the wearer’s foot when the insole is positioned in the article of footwear and the article of footwear is worn.

### BACKGROUND

Articles of footwear often include both an upper and a sole. The upper encloses a wearer’s foot, and in some circumstances provides support for the foot during motion. The sole generally provides traction, protection, and also can support the foot. Typically, an article of footwear also includes an insole placed within the upper between the wearer’s foot and the sole to provide additional comfort as well as increased performance for various activities.

Articles of footwear typically do not include lights. For footwear having lights prior art patent documents are typically directed to footwear where the light is directed outward to produce a warning effect or enhance the outward visual effect of the shoe. For example, US 2006/0221596 A1 discloses a luminescent panel suitable for decorating shoes. The patent document discloses fiber optic fibers woven with nylon fibers into a warp and weft knitted fabric, to form a luminescent panel in the shape of leaflet. The luminescent panel is not, however, depicted in an area of the shoe that would carry a load of the wearer when the shoe is being worn.

US 2008/0308748 A1 depicts a footwear sterilization and disinfection apparatus and method using UV-C light to sterilize or disinfect the inside of the footwear. This patent document discloses an insole having a fiber optic weave embedded in the insole. The fiber optic weave is used to distribute light throughout the footwear or shoe when it is not being worn. The source of the UV-C light could be mercury vapor bulbs contained within a fixture that connects to a receptacle in either the heel or the toe of the footwear and projects the UV-C light into the fiber optic weave to distribute the light to the remainder of the footwear.

The aforementioned prior art documents fail to adequately consider protecting the optical fiber from loads that may be applied if the fiber was incorporated into the insole of an article of footwear.

### SUMMARY

In view of the foregoing, an insole for an article of footwear includes an insole support member and a side-emitting optical fiber fixed with respect to the insole support member. The insole support member is made from a cushioning material and includes a top surface and a bottom surface. The side-emitting optical fiber is configured to project radiation having a therapeutic wavelength along the length of and outwardly through a side of the side-emitting optical fiber and toward a wearer’s foot when the insole is positioned in the article of footwear and the article of footwear is worn. At least a majority of an uppermost surface of the side-emitting optical fiber is disposed beneath a foot-contacting surface that is in contact with the wearer’s foot when the insole is positioned in the article of footwear and the article of footwear is worn.

### BRIEF DESCRIPTION OF THE DRAWINGS

FIG. 1 is an upper perspective view of an article of footwear with an upper shown in phantom.

FIG. 2 is a cross-sectional view of a side-emitting optical fiber for use with an insole depicted in FIG. 1.

FIG. 3 is an exploded view of an example of an insole that can be inserted into the article of footwear depicted in FIG. 1.

FIG. 4 a cross-sectional view of a side-emitting light tube for use with some examples of insoles described herein.

FIG. 5 is an assembled view of another example of an insole that can be inserted into the article of footwear depicted in FIG. 1.

FIG. 6 is a cross-sectional view taken through line 6-6 of FIG. 5.

FIG. 7 is an alternative side-emitting light tube shown in cross section.

FIG. 8 is an exploded view of another example of an insole that can be inserted into the article of footwear depicted in FIG. 1.

FIG. 9 is an exploded view of another example of an insole that can be inserted into the article of footwear depicted in FIG. 1.

FIG. 10 is a cross-sectional view taken through an assembled portion of the insole example depicted in FIG. 9.

FIG. 11 is an exploded view of another example of an insole that can be inserted into the article of footwear depicted in FIG. 1.

FIG. 12 is an exploded view of another example of an insole that can be inserted into the article of footwear depicted in FIG. 1.

FIG. 13 is a cross-sectional view taken through an assembled portion of the insole example depicted in FIG. 12.

FIG. 14 is a plan view of a light pod for use with the insoles mentioned above.

FIG. 15 is a schematic depiction of the light pod of FIG. 14.

FIG. 16 is an exploded view of the article of footwear depicted in FIG. 1.

FIG. 17 is a side view of an article of footwear and the light pod.

FIG. 18 is an upper perspective view of an insole assembly including an extension.

FIG. 19 is an upper view of an article of footwear with the insole assembly of FIG. 18.

### DETAILED DESCRIPTION

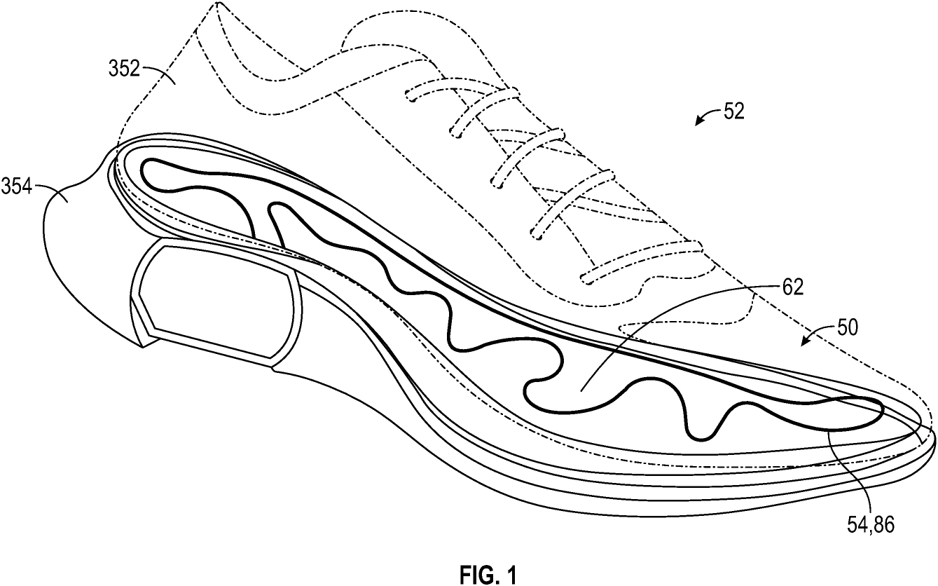

FIG. 1 depicts an insole 50 for an article of footwear 52. The insole 50 includes provisions for operating as a light (or radiation) delivery system in that it is configured to project light (or radiation) toward the foot of the wearer of the article of footwear 52. Light having a wavelength between 630 nm and 900 nm has been found beneficial to increase blood flow, may provide ameliorative effects with regard to inflammation, and can be beneficial in the treatment of diabetic neuropathy, and as such can be referred to as light having a therapeutic wavelength. The insole 50 may be configured to project light at wavelengths between 630 nm and 900 nm, which also may have a therapeutic effect, including, but not limited to infrared (IR) light. Even though FIG. 1 depicts a particular style of footwear, the insole 50 may be adapted for placement in any kind of footwear, including, but not limited to: running shoes, hiking boots, soccer shoes, football shoes, sneakers, rugby shoes, basketball shoes, baseball shoes as well as other kinds of non-athletic shoes, including, but not limited to: dress shoes, loafers, sandals, and boots.

The insole 50 includes a side-emitting optical fiber 54 configured to project radiation along the length of and outwardly through a side of the side-emitting optical fiber. The side-emitting optical fiber 54 is positioned within the article of footwear 52 so as to direct light toward a wearer’s foot when the insole 50 is positioned in the article of footwear 52 and the article of footwear 52 is worn.

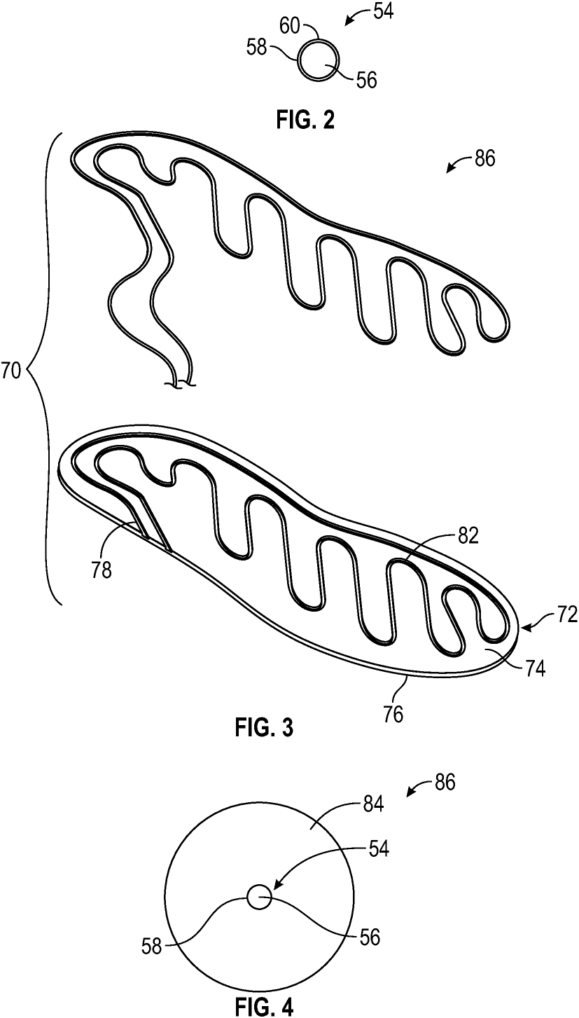

With reference to FIG. 2, the side-emitting optical fiber 54 includes a core 56 and cladding 58 surrounding the core 56. The core 56 in the illustrated embodiment is made from a synthetic polymeric material, e.g., a high-purity polymethyl methacrylate (PMMA). The cladding 58 in the illustrated embodiment is made from a fluorinated polymer. Unlike typical optical fibers, for example an optical fiber used to transmit light between two ends of the optical fiber in fiber-optic communications, the side-emitting optical fiber 54 transmits light outwardly along the length of the side-emitting optical fiber 54. For example, the cladding 58 can be etched, either mechanically or chemically, to allow light to escape outwardly through the side of the side-emitting optical fiber 54 along the length of the side-emitting optical fiber 54.

Different from shoes provided with a woven luminescent panel in which fiber optic fibers are woven with nylon fibers or shoes provided with a fiber optic weave in the insole, the side-emitting optical fiber 54 is positioned such that at least a majority of an uppermost surface 60 of the side-emitting optical fiber 54 is disposed beneath a foot-contacting surface 62, which is in contact with the wearer’s foot when the insole 50 is positioned in the article of footwear 52 and the article of footwear 52 is worn. In many examples described below at least 90% of the uppermost surface 60 of the side-emitting optical fiber 54 is disposed beneath a foot-contacting surface 62. Offsetting the side-emitting optical fiber 54 from the foot-contacting surface 62 provides protection to the side-emitting optical fiber 54 from a force being applied by the wearer’s foot when the insole 50 is positioned in the article of footwear 52 and the article of footwear 52 is worn.

Because of the position of the side-emitting optical fiber 54 within the article of footwear 52, a protective layer, examples of which will be described in more detail below, can also be positioned between the side-emitting optical fiber 54 and the foot-contacting surface 62. The protective layer is light-transmissive and is configured to inhibit damage to the side-emitting optical fiber 54 from the force being applied by the wearer’s foot when the insole 50 is positioned in the article of footwear 52 and the article of footwear 52 is worn.

FIG. 3 depicts a first example of an insole 70 including an insole support member 72 having a top surface 74 and a bottom surface 76. The top surface 74 faces towards a wearer’s foot when the insole 70 is positioned in the article of footwear 52 and the article of footwear 52 is worn. The insole support member 72 can be made from a cushioning material, e.g., an EVA resin foam, a soft polyethylene foam and/or a polyurethane (PU) material, which may be referred to as popcorn PU. Although not shown in detail, the insole support member 72 may include a layered or partially layered structure similar to known inserts, and the materials from which the layers are made may have a different hardness.

The insole support member 72 includes a channel 78 (multiple similarly shaped channels could also be provided) extending downwardly from the top surface 74 toward the bottom surface 76. The channel 78 can define a channel floor 82. When provided, the channel floor 82 is offset downwardly from the top surface 74. In some circumstances the channel floor 82 can be offset upwardly from the bottom surface 76. In other circumstances, the channel 78 may extend entirely through the insole support member 72.

In the first example of the insole 70, and with reference to FIG. 4, the protective layer is a jacket 84 that at least partially surrounds the side-emitting optical fiber 54. Accordingly, the side-emitting optical fiber 54 and the protective layer, i.e., the jacket 84 in this first example, are parts of a side-emitting light tube 86. The side-emitting light tube 86 and thus the side-emitting optical fiber 54 in this first example are received in the channel 78 and are fixed with respect to the insole support member 72 such that movement of the insole support member 72 results in movement of the side-emitting optical fiber 54 and the side-emitting light tube 86.

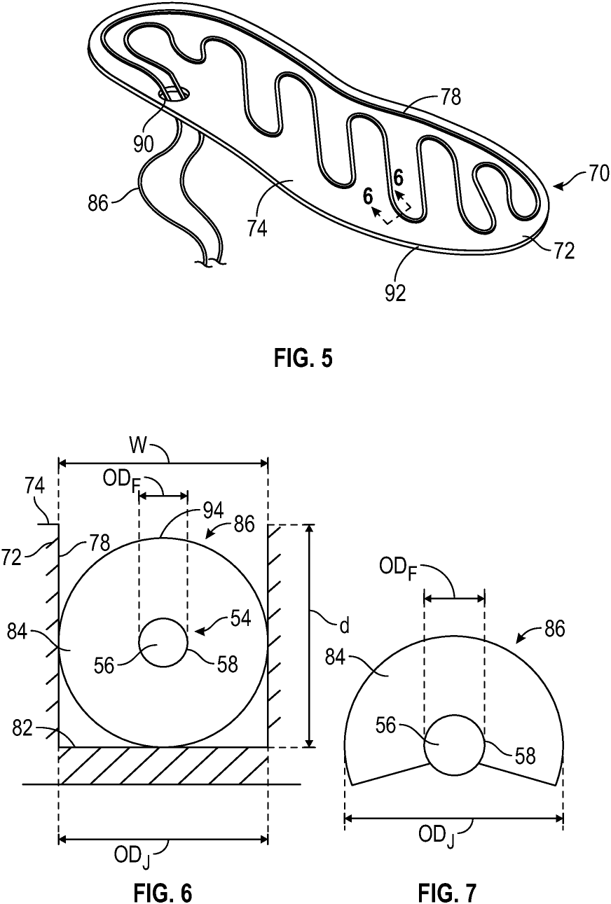

FIG. 5 depicts the insole 70 in an assembled state with the side-emitting light tube 86 received in the channel 78, although the insole support member 72 has been slightly altered to include an opening 90 and the channel 78 no longer extends to a peripheral edge 92 of the insole support member 72. With reference to FIG. 6, when provided, the channel floor 82 can be offset downwardly from the top surface 74 to provide the channel 78 with a depth d between 0.25 mm and 4 mm. When the channel 78 extends entirely through the insole support member 72, the channel 78 can have a depth d measured downwardly from the top surface 74 even greater than 4 mm. The channel 78 can have a width w measured perpendicular to the depth d between 0.25 mm and 4 mm. In FIG. 6 the channel floor 82 is depicted as residing in a plane, however, the channel floor 82 could be curved.

The jacket 84 in the illustrated embodiment is made from silicone, which can be light-transmissive, having a durometer of at least 50A, and preferably at least 65A. The jacket 84 can be extruded over the side-emitting optical fiber 54 such that the jacket 84 surrounds the side-emitting optical fiber 54 and is in contact with the cladding 58. Even though the jacket 84 is in contact with the cladding 58 so that no or a very small air gap is provided between them, the side-emitting optical fiber 54 may be movable with respect to the jacket 84 in an axial direction, which is parallel with a longest dimension of the side-emitting light tube 86. Allowing for movement of the side-emitting optical fiber 54 with respect to the jacket 84 can be helpful in preventing damage to the side-emitting optical fiber 54 when the side-emitting light tube 86 is bent.

The maximum outer dimension of the jacket 84, which will be referred to as an outer diameter (OD_{J}) although the jacket 84 need not be circular in cross-section but instead could be U-shaped (see FIG. 7), for example, is at least four times, and preferably at least six times, the outer diameter (OD_{F}) of the side-emitting optical fiber 54. As an example, the outer diameter (OD_{F}) of the side-emitting optical fiber 54 can be 0.25 mm and the outer diameter (OD_{J}) of the jacket 84 between 1.0 mm and 1.5 mm. The side-emitting optical fiber 54 could have a smaller outer diameter (OD_{F}). As another example, the outer diameter (OD_{F}) of the side-emitting optical fiber 54 can be 0.5 mm with the outer diameter (OD_{J}) of the jacket 84 being between two times to three times greater than the maximum outer dimension (OD_{F}) of the side-emitting optical fiber 54. Having the outer diameter (OD_{F}) of the side-emitting optical fiber 54 larger than 0.5 mm with the relatively larger jacket 84 can result in the side-emitting light tube 86 being too large in cross-section making it difficult to incorporate into the insole 70 in an aesthetic and practically functional manner. Providing the jacket 84 with the maximum outer dimension (OD_{J}) that is between two times to six times greater that the outer diameter (OD_{F}) of the side-emitting optical fiber 54 protects the side-emitting optical fiber 54 from the force being applied by the wearer’s foot when the insole 70 is positioned in the article of footwear 52 and the article of footwear 52 is worn.

During assembly, the side-emitting light tube 86 can be pressed into the channel 78 so that the side-emitting light tube 86 is received in the channel 78. With particular reference to FIG. 6, an uppermost surface 94 on the side-emitting light tube 86 may be offset beneath the top surface 74, which in the first example can coincide with the foot-contacting surface 62 (FIG. 1). As illustrated in FIG. 6, the uppermost surface 94 on the side-emitting light tube 86 is offset beneath the top surface 74. The maximum outer dimension (OD_{J}) of the jacket 84 may be less than the depth (d) of the channel 78. The maximum outer dimension (OD_{J}) of the jacket 84 may also be less than or equal to the width of the channel 78. Where the maximum outer dimension (OD_{J}) of the jacket 84 is equal to the width of the channel 78, the side-emitting light tube 86 may be retained within the channel 78 via frictional engagement with the insole support member 72.

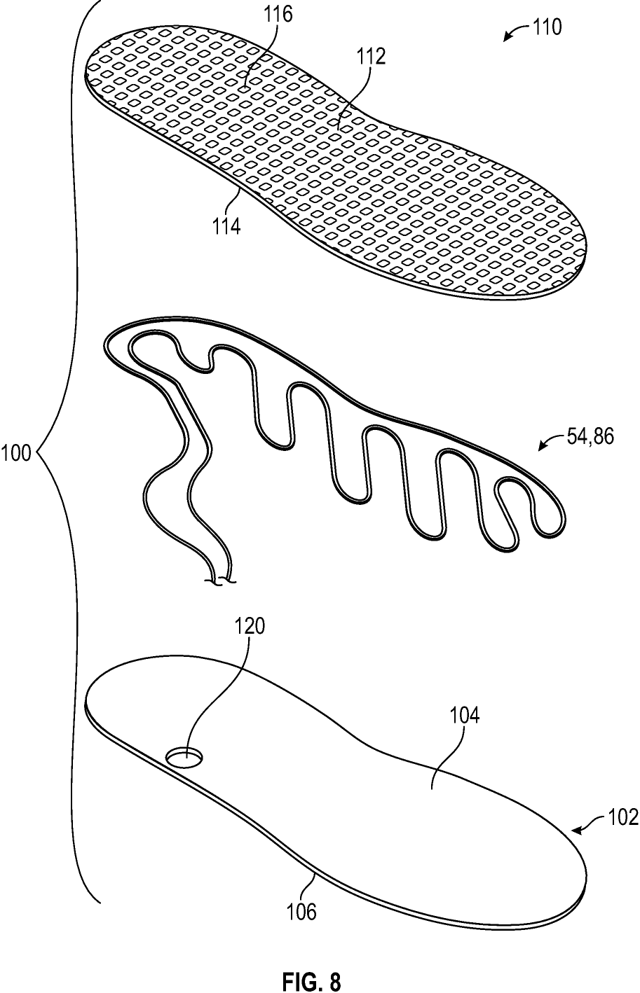

FIG. 8 depicts a second example of an insole 100 including an insole support member 102 having a top surface 104 and a bottom surface 106. The top surface 104 faces towards a wearer’s foot when the insole 100 is positioned in the article of footwear 52 and the article of footwear 52 is worn. The insole support member 102 in the second example is made from a cushioning material, e.g., an EVA resin foam, a soft polyethylene foam and/or a polyurethane (PU) material, which may be referred to as popcorn PU. Although not shown in detail, the insole support member 102 may include a layered or partially layered structure similar to known inserts, and the materials from which the layers are made may have a different hardness. In the second example, the insole support member 102 need not include a channel.

In the second example of the insole 100, the protective layer is a protective liner 110 positioned over the top surface 104 and at least partially covering the side-emitting optical fiber 54. As illustrated in FIG. 8, the protective liner 110 has an overall shape and periphery matching an overall shape and periphery of the top surface 104 of the insole support member 102, however, the protective liner 110 can take other configurations. The protective liner 110 is light transmissive. For example, the protective liner 110 can be made from a light transmissive material. Examples of materials from which the protective liner 110 can be made include elastomers or plastics, a more particular example being silicone. Also, the elastomers or plastics can have a durometer of at least 50A, and preferably at least 65A. The protective liner 110 may also be made from an opaque material when provided with apertures 116 to allow light to travel through. The protective liner 110 includes a liner upper surface 112, which can coincide with the foot-contacting surface 62 (FIG. 1), and a liner lower surface 114 that contacts the top surface 104 and the side-emitting optical fiber 54.

The thickness, which is measured between the liner upper surface 112 and the liner lower surface 114, of the protective liner 110 is at least two times, and preferably at least three times, the outer diameter (OD_{F}) of the side-emitting optical fiber 54 when the side-emitting optical fiber 54 is “unjacketed,” i.e., not provided with the jacket 84 depicted in FIG. 4. As an example, the outer diameter (OD_{F}) of the side-emitting optical fiber 54 can be 0.25 mm and the thickness of the protective liner 110 at least between 0.5 mm and 0.75 mm.

During assembly, the side-emitting optical fiber 54 is laid out on the top surface 104 in the desired pattern. If an opening 120 is provided, then a portion of the side-emitting optical fiber 54 can be passed through the opening 120. The protective liner 110 is then placed on the top surface 104 and the side-emitting optical fiber 54. If desired, the protective liner 110 is adhered, sewn or affixed to the insole support member 102.

With continued reference to FIG. 8, in lieu of using the “unjacketed” side-emitting optical fiber 54, the side-emitting light tube 86, which includes the side-emitting optical fiber 54 and the jacket 84, could be laid out on the top surface 104 with a portion thereof passing through opening 120 (if provided). If the side-emitting light tube 86 were used in place of the “unjacketed” side-emitting optical fiber 54, then the thickness of the protective liner 110 could be reduced; however, the thickness of the protective liner 110 should be thick enough and/or the hardness (shore A) of the material from which the protective liner 110 is made specified so to inhibit undulations from forming in the areas of the protective liner 110 that cover the side-emitting light tube 86.

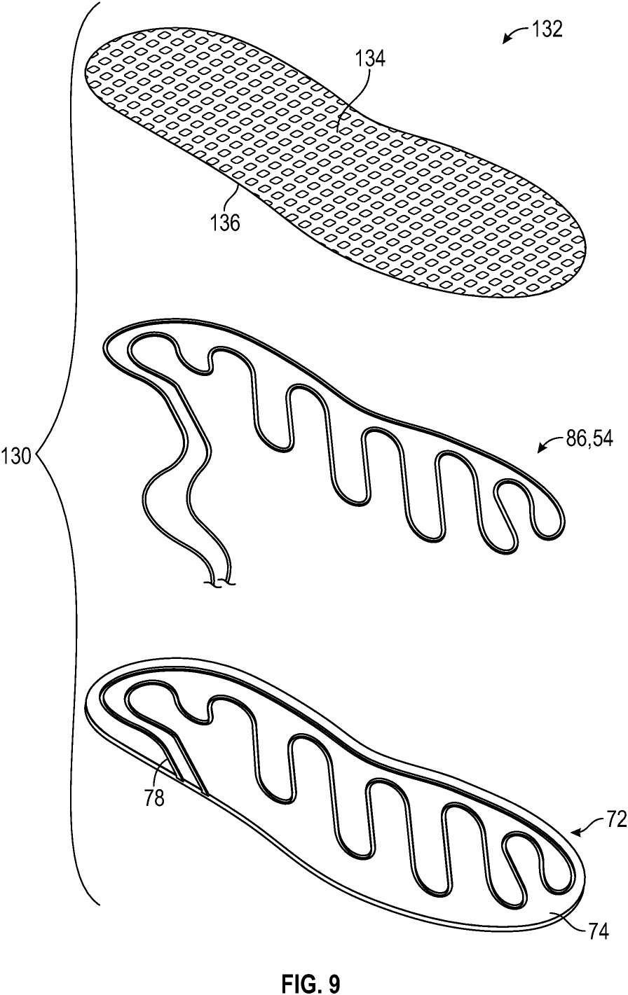

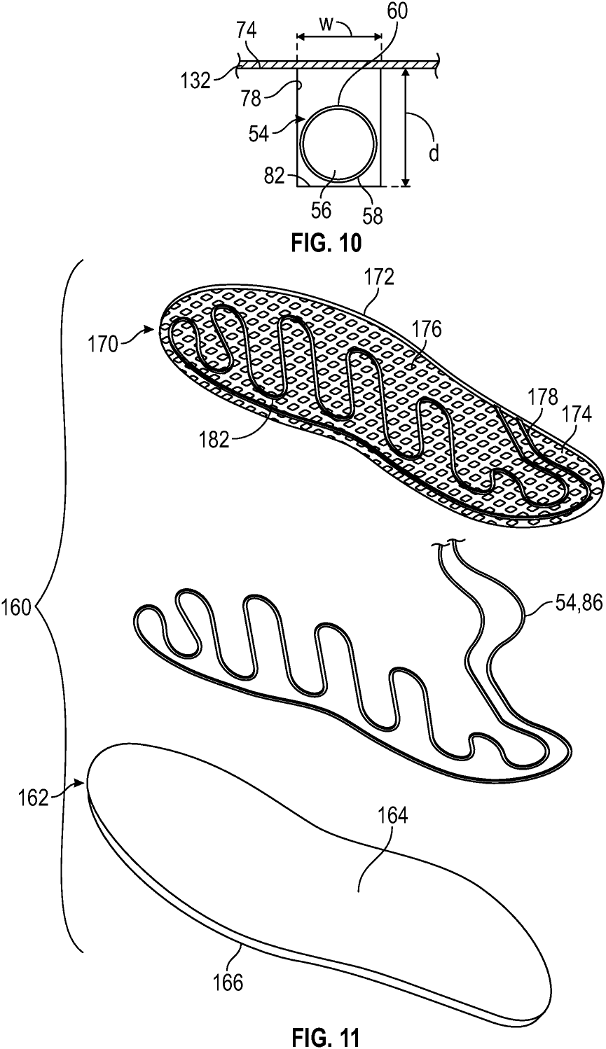

FIG. 9 depicts a fourth example of an insole 130 including the insole support member 72 and the side-emitting light tube 86 described above with reference to FIGS. 3 - 7 and an insole liner 132. The side-emitting light tube 86 is received in the channel 78 in the same manner as the first example described above, and therefore is not described again in particularity for the sake of brevity.

The insole liner 132 is light-transmissive; however, it need not be as robust as the protective liner 110 described above with reference to FIG. 8 when the side-emitting light tube 86 is employed. The insole liner 132 may comprise a cloth or fabric material in some embodiments. In other embodiments, the insole liner 132 may comprise a mesh (fabric or plastic) material and/or a plastic film. The insole liner 132 includes an upper side 134, which can be coincident with the foot-contacting surface 62 (FIG. 1), and a lower side 136. The lower side 136 of the insole liner 132 is disposed on the top surface 74 of the insole support member 72. The upper side 134 of insole liner 132 can be coincident with the foot-contacting surface 62 (FIG. 1). The insole liner 132 may adhered, sewn or otherwise affixed to the top surface 74 of the insole support member 72. In the illustrated embodiment, the periphery of the insole liner 132 matches the periphery of the insole support member 72. This can allow the insole 130 to have an overall shape with a periphery conforming to a traditional footbed of an article of footwear. If desired, however, the insole liner 132 need not match the periphery of the insole support member 72, but instead may only cover the side-emitting light tube 86 on the insole support member 72. As such, the insole liner 132 can be light-transmissive in at least one area above the side-emitting light tube 86 and positioned at least partially over the side-emitting light tube 86 and the top surface 74. In other words, the entirety of the top surface 74 and the entirety of the side-emitting light tube 86 need not be covered by the insole liner 132. The insole liner 132 may also retain the side-emitting light tube 86 to the top surface 74 of the insole support member 72.

With continued reference to FIG. 9, in lieu of using the side-emitting light tube 86, which includes the side-emitting optical fiber 54 and the jacket 84, the “unjacketed” side-emitting optical fiber 54 could be inserted into the channel 78. In the fifth example, the uppermost surface 60 on the side-emitting optical fiber 54 is offset beneath the top surface 74. The maximum outer dimension (OD_{F}) of the side-emitting optical fiber 54 is less than the depth (d) of the channel 78 to ensure that the uppermost surface 60 on the side-emitting optical fiber 54 is offset beneath the top surface 74, which is coincident with the foot-contacting surface 62. The maximum outer dimension (OD_{F}) of the side-emitting optical fiber 54 is also be less than or equal to the width of the channel 78.

FIG. 11 depicts a sixth example of an insole 160 having an insole support member 162 similarly constructed to the insole support member 102 depicted in FIG. 8, but lacking the opening depicted in FIG. 8. The insole support member 162 in the sixth example includes a top surface 164 and a bottom surface 166. The top surface 164 faces towards a wearer’s foot when the insole 160 is positioned in the article of footwear 52 and the article of footwear 52 is worn. Like the insole support member 102 depicted in FIG. 8, the insole support member 162 also lacks channels.

In the sixth example of the insole 160, the protective layer is a protective liner 170 positioned over the top surface 164 and at least partially covers the side-emitting optical fiber 54. The protective liner 170 includes a liner upper surface 172, which can coincide with the foot-contacting surface 62 (FIG. 1), and a liner lower surface 174 that contacts the top surface 164 when the insole 160 is assembled. As illustrated in FIG. 11, the protective liner 170 has an overall shape and periphery matching an overall shape and periphery of the top surface 164 of the insole support member 162, however, the protective liner 170 can take other configurations. The protective liner 170 is light transmissive. For example, the protective liner 170 can be made from a light transmissive material. Examples of materials from which the protective liner 170 can be made include elastomers or plastics. Also, the elastomers or plastics can have a durometer of at least 50A, and preferably at least 65A. The protective liner 170 may also be made from an opaque material when provided with apertures 176 to allow light to travel through.

The protective liner 170 includes a channel 178 (multiple similarly shaped channels could also be provided) extending upwardly from the liner lower surface 174 toward the liner upper surface 172. The channel 178 defines a channel roof 182. A thickness measured between the liner upper surface 172 and the channel roof 182 is at least two times, and preferably at least three times, the outer diameter (OD_{F}) of the side-emitting optical fiber 54 when the side-emitting optical fiber 54 is “unjacketed,” i.e., not provided with the jacket 84 depicted in FIG. 4. As an example, the outer diameter (OD_{F}) of the side-emitting optical fiber 54 can be 0.25 mm and the thickness measured between the liner upper surface 172 and the channel roof 182 is at least between 0.5 mm and 0.75 mm.

With continued reference to FIG. 11, in lieu of using the “unjacketed” side-emitting optical fiber 54, the side-emitting light tube 86, which includes the side-emitting optical fiber 54 and the jacket 84, could be inserted into the channel 178. If the side-emitting light tube 86 were used in place of the “unjacketed” side-emitting optical fiber 54, then the thickness measured between the liner upper surface 172 and the channel roof 182 could be reduced; however, the thickness measured between the liner upper surface 172 and the channel roof 182 should be thick enough and/or the hardness (shore A) of the material from which the protective liner 170 is made is specified so to inhibit undulations from forming in the areas of the protective liner 170 that cover the side-emitting light tube 86.

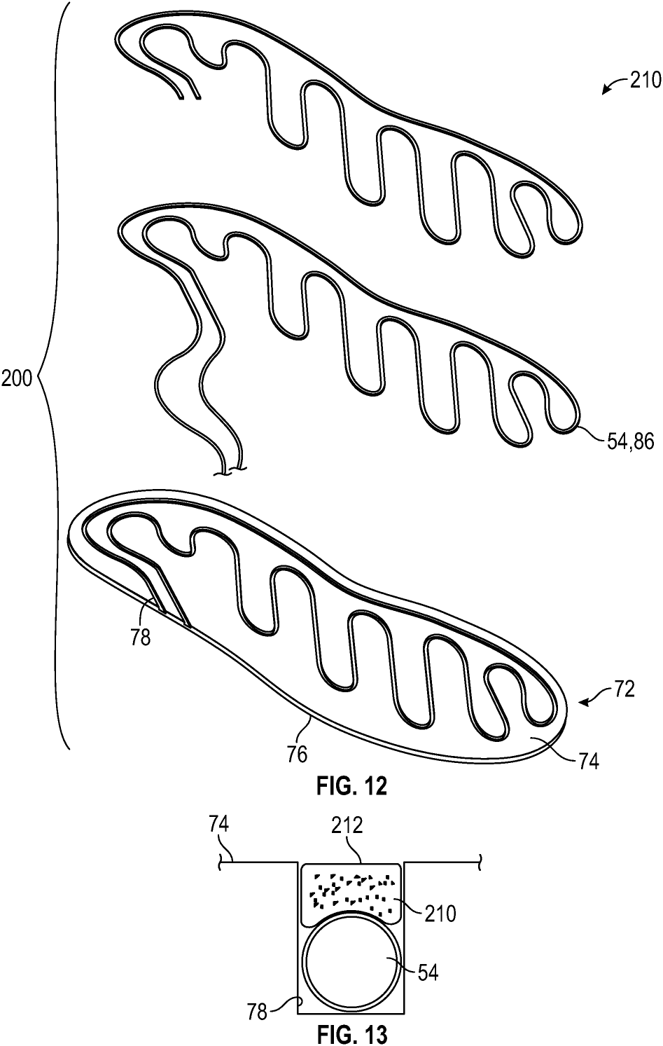

FIG. 12 depicts an eighth example of an insole 200 where the insole support member 72, which is more particularly descried above, includes the top surface 74, the bottom surface 76 and the channel 78. In the eighth example either the side-emitting light tube 86, which includes the side-emitting optical fiber 54 and the jacket 84, or the “unjacketed” side-emitting optical fiber 54 is inserted into the channel 78.

In this example, the protective layer is a protective insert 210 that is also received in the channel 78. At least a portion of the protective insert 210 is positioned between the side-emitting optical fiber 54 and the foot-contacting surface 62 (FIG. 1), which can also coincident with the top surface 74. The protective insert 210 is made from a light transmissive material. Examples of materials from which the protective insert 210 can be made include elastomers or plastics, a more particular example being silicone. Also, the elastomers or plastics can have a durometer of at least 50A, and preferably at least 65A.

FIG. 13 depicts the protective insert 210 placed into the channel 78 above the side-emitting optical fiber 54 with a protective insert top surface 212 substantially flush with the top surface 74 of the insole support member 72. The protective insert 210 can be in the form of an already formed (set) silicone (or similar material) strip that is the approximate length and width of the channel 78, and this formed strip can be pressed into the channel 78. Alternatively, the protective insert 210 can be in the form of a flowable silicone (or similar material) that is poured or injected into the channel 78 over the side-emitting optical fiber 54.

Although several different examples of insoles have been described above with reference to different figures, aspects of one example could be used with others.

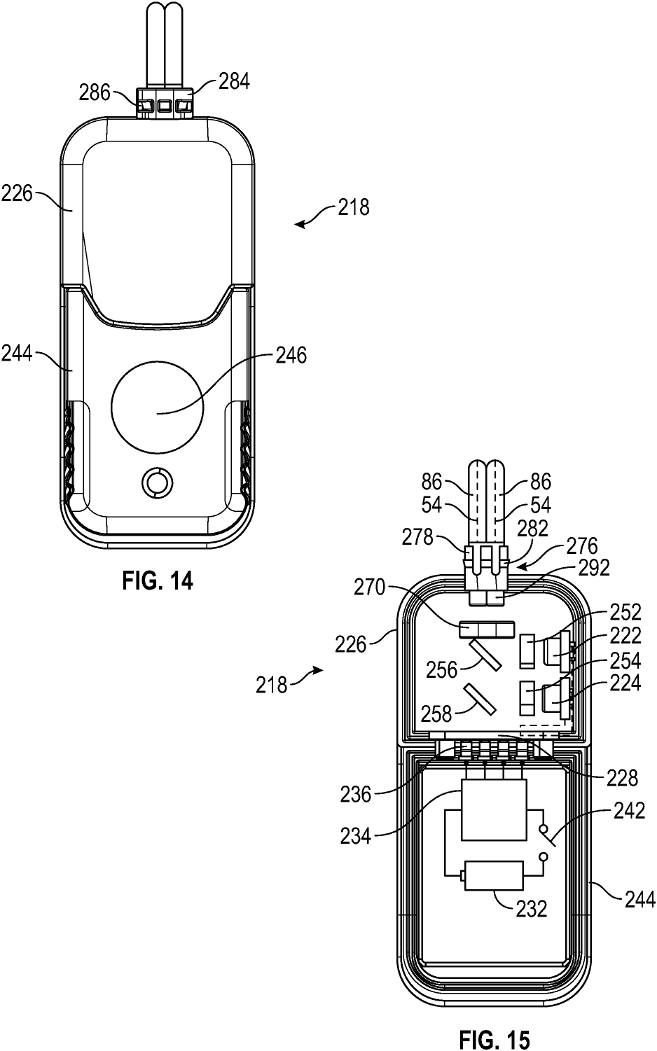

With reference to FIG. 14, in each of the insole examples mentioned above, the side-emitting optical fiber 54, which can be provided as a component of the side-emitting light tube 86 or as an “unjacketed” side-emitting optical fiber 54, is optically connected with a light pod 218. FIG. 15 is a schematic depiction of the light pod 218 in which an optical fiber light source, which in the illustrated embodiment includes a red laser diode 222 that emits red light (about 660 nm) and an infrared (IR) laser diode 224 that emits IR radiation (about 820-850 nm), are both received in the light source housing 226. Other types of light sources, e.g., an LED, could be used instead of the laser diodes. A light source electrical contact 228, which can include electrical receptacles and appropriate circuity (not depicted) is in electrical communication (depicted schematically) with the red laser diode 222 and the IR laser diode 224.

A power source, such as a battery 232 is in electrical communication with a controller 234, which is electrically connected with power source electrical contacts 236. The controller 234 can be an appropriate integrated circuit, for example. The controller 234 is in electrical communication with a switch 242 that can control power delivery to the controller 234, the red laser diode 222 and the IR laser diode 224. The battery 232, the controller 234 and the switch 242 can be received in a power source housing 244, and an actuator 246 (FIG. 14), e.g., a button, can be accessible to a user on the outside of the power source housing 244 for operating the switch 242. The power source electrical contacts 236, which can be pogo pins, can connect with the light source electrical contact 228 when the power source housing 244 is connected with the light source housing 226.

The red laser diode 222 and the IR laser diode 224 optically couple to the side-emitting optical fiber 54 so as to project light into the side-emitting optical fiber 54. A first lens 252 is provided in the light source housing 226 and cooperates with the red laser diode 222. A second lens 254 is also provided in the light source housing 226 and cooperates with the IR laser diode 224. The first lens 252 directs the light emanating from the red laser diode 222 toward a first mirror 256. The second lens 254 directs the radiation emanating from the IR laser diode 224 toward a second mirror 258. The first mirror 256 reflects the red light from the first lens 252 and redirects it toward a beam splitter 270. The second mirror 258 reflects the IR light from the second lens 254 and redirects it toward the beam splitter 270. The first mirror 256 is configured to allow IR radiation to pass though it toward the beam splitter 270. The beam splitter 270 is configured to split both the red light from the first mirror 256 and the IR radiation from the second mirror 258 into each end of the side-emitting optical fiber 54.

To facilitate connecting each end of the side-emitting light tube 86 with the light pod 218, a ferrule mount 276 having resilient fingers 278 each with a respective barb 282 is provided at the ends of the side-emitting light tube 86. With reference back to FIG. 14, the light source housing 226 includes a receptacle 284 that receives the ferrule mount 276. The receptacle 284 includes openings 286 that each receive a respective barb 282 to connect the ferrule mount 276 with the receptacle 284. With reference back to FIG. 15, each end of the side-emitting light tube 86 attaches with a respective ferrule 292, which includes an opening (not visible) through which light or radiation can pass into the side-emitting optical fiber 54. The light pod 218 is configured to allow two different types of radiation to enter each end of the side-emitting optical fiber 54.

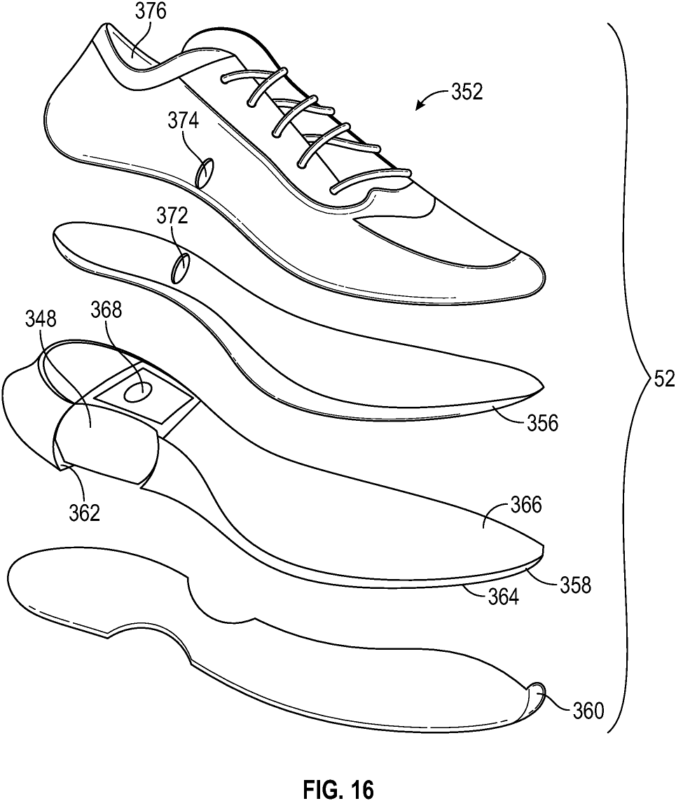

FIG. 16 depicts the article of footwear 52 including a light pod 348, which can have a similar construction as the light pod 218 described with reference to FIGS. 14 and 15. The article of footwear 52 can include an upper 352 and a sole 354 (see FIG. 1). The sole 354 can be made up of a midsole, which can include an upper midsole 356, a lower midsole 358, and an outsole 360, which is typically made of a durable material, e.g., rubber. The sole 354 is generally designed to provide traction, protection, and also to support the foot. In some embodiments, the upper midsole 356 may be omitted.

The sole 354, and more particular to the embodiment depicted in FIG. 16, the lower midsole 358 defines a light pod cavity 362 in which the light pod 348 is located. In FIG. 16, the light pod cavity 362 is located near where a wearer’s heel would land when the article of footwear 52 is being worn; however, the light pod cavity 362 could be positioned elsewhere, e.g., where a wearer’s arch would land when the article of footwear 52 is being worn. The light pod cavity 362 can extend from a lower surface 364 to an upper surface 366 of the lower midsole 358, as shown in FIG. 16. Alternatively, the light pod cavity 362 can extend from the lower surface 364 toward, but not all the way to, the upper surface 366. If the light pod cavity 362 does not extend through to the upper surface 366 of the lower midsole 358, then an opening (not shown) can be provided aligned with a connection opening 368 in the light pod 348. If the upper midsole 356 is provided, then it can also include an opening 372 that aligns with the connection opening 368 in the light pod 348 when the article of footwear 52 is finally assembled. Also, if the upper 352 is provided with a lower panel or lower member, this lower panel or member may also be provided with an opening 374 that aligns with the connection opening 368 in the light pod 348 when the article of footwear 52 is finally assembled. When provided with the lower panel or member, the upper 352 includes the opening 374, which can be a slot or a hole extending through the upper 352 that is distinct and offset from an opening 376 in the article of footwear 52 that receives the wearer’s foot when the article of footwear 52 is put on. Any example of the insoles described above can be inserted into the article of footwear 52 and the side-emitting light tube 86 or the “unjacketed” side-emitting optical fiber 54 can pass through the openings 372 and 374 toward the connection opening 368 in the light pod 348 to connect with the light pod 348 in a similar manner to that described above with reference to FIGS. 14 and 15.

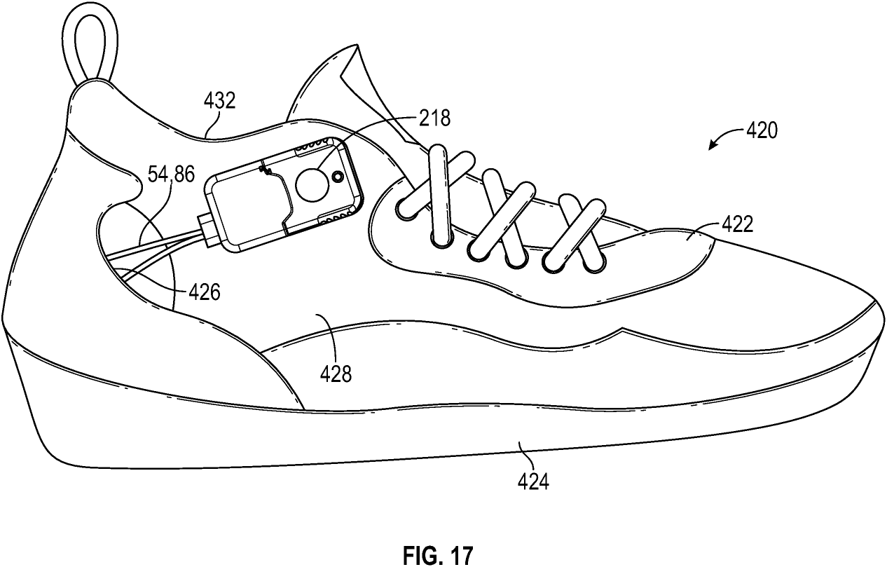

FIG. 17 depicts an article of footwear 420 having an upper 422 and a sole 424. Any of the aforementioned insoles can be placed within the upper 422 between the wearer’s foot and the sole 424 in a similar manner to a conventional insole. The upper 422 also includes a passage 426, which can be a slot or a hole extending through the upper 422 from inside the upper 422 to an outer surface 428 of the article of footwear 420. The passage 426 in the embodiment illustrated in FIG. 17 is distinct and offset from an opening 432 in the article of footwear 420 that receives the wearer’s foot when the article of footwear 420 is put on. The light pod 218 is configured to attach to the outer surface 428 of the article of footwear 420, and more particularly in the illustrated embodiment to the outer surface 428 of the upper 422. The side-emitting light tube 86 or the “unjacketed” side-emitting optical fiber 54 extends through the passage 426.

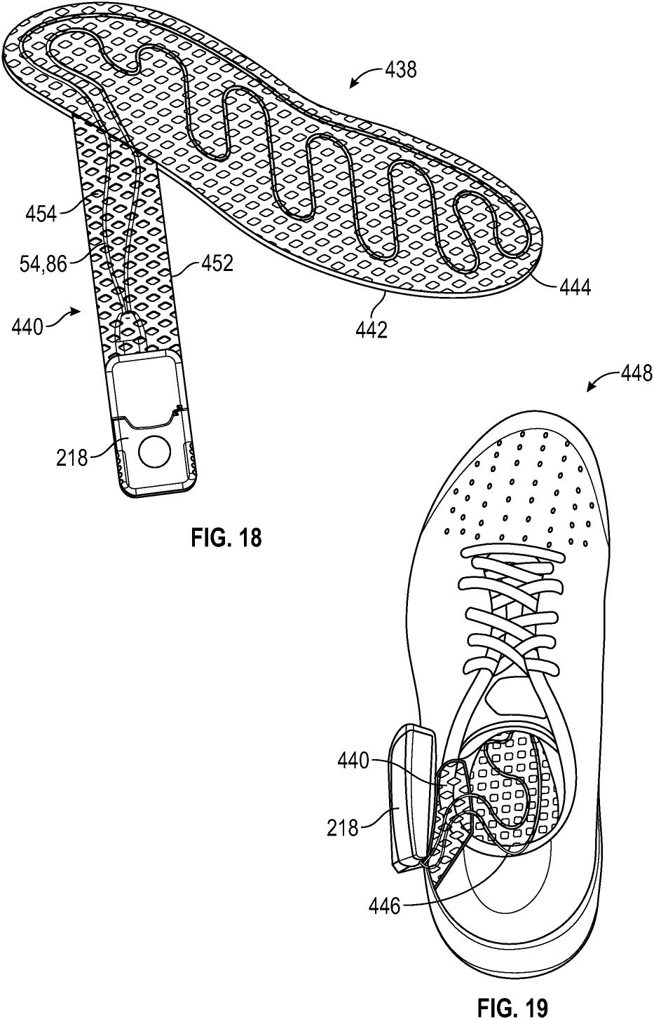

FIG. 18 depicts the insole assembly 438 further including an extension 440. The extension 440 extends from a periphery 442 of an insole support member 444, which could be an insole support member similar to any of those described above. With reference to FIG. 19, the extension 440 is configured to extend outwardly from the opening 446 in an article of footwear 448 configured to receive a wearer’s foot. The article of footwear 448 can be a conventional article of footwear. The extension 440 can be made from a strip 452 of material that is a material similar to the material from which the insole support member 444 is made. An extension liner 454, which can be made from a material similar to the material from which the insole liner 132 (FIG. 9) is made, can cover an upper surface of the strip 452. The side-emitting light tube 86 or the “unjacketed” side-emitting optical fiber 54 can be fixed to the extension 440, e.g. the strip 452 may or may not have channels, and side-emitting light tube 86 or the “unjacketed” side-emitting optical fiber 54 can be received in the channels. Also, the extension liner 454 can cover the side-emitting light tube 86 or the “unjacketed” side-emitting optical fiber 54. The light pod 218 may be fixed to the extension 440.

It will be appreciated that various of the above-disclosed embodiments and other features and functions, or alternatives or varieties thereof, may be desirably combined into many other different systems or applications. Also that various presently unforeseen or unanticipated alternatives, modifications, variations or improvements therein may be subsequently made by those skilled in the art which are also intended to be encompassed by the following claims.

1. An insole for an article of footwear comprising:
an insole support member made from a cushioning material and having a top surface and a bottom surface; and
a side-emitting optical fiber fixed with respect to the insole support member, the side-emitting optical fiber configured to project radiation having a therapeutic wavelength along the length of and outwardly through a side of the side-emitting optical fiber and toward a wearer’s foot when the insole is positioned in the article of footwear and the article of footwear is worn, wherein at least a majority of an uppermost surface of the side-emitting optical fiber is disposed beneath a foot-contacting surface that is in contact with the wearer’s foot when the insole is positioned in the article of footwear and the article of footwear is worn.

2. The insole of claim 1, wherein at least 90% of the uppermost surface of the side-emitting optical fiber is disposed beneath the foot-contacting surface.

3. The insole of claim 1, wherein the insole support member includes a channel, and the side-emitting optical fiber is received in the channel.

4. The insole of claim 3, wherein the channel defines a channel floor offset downwardly from the top surface and a lowermost portion of the channel floor is offset downwardly from the top surface between 0.25 mm to 4 mm.

5. The insole of claim 1, further comprising a protective layer, which is light-transmissive, positioned between the side-emitting optical fiber and the foot-contacting surface, the protective layer being configured to inhibit damage to the side-emitting optical fiber from a force being applied by the wearer’s foot when the insole is positioned in the article of footwear and the article of footwear is worn.

6. The insole of claim 5, wherein the side-emitting optical fiber and the protective layer are parts of a side-emitting light tube in which the protective layer is a jacket that at least partially surrounds the side-emitting optical fiber.

7. The insole of claim 6, wherein the insole support member includes a channel, and the side-emitting light tube is received in the channel.

8. The insole of claim 7, wherein an uppermost surface on the side-emitting light tube is beneath the foot-contacting surface.

9. The insole of claim 6, wherein the jacket is circular in cross section.

10. The insole of claim 6, wherein the jacket is made from a light transmissive elastomer or plastic.

11. The insole of claim 10, wherein the elastomer or plastic has a durometer of at least 50A.

12. The insole of claim 6, further comprising an insole liner, which is light-transmissive, positioned at least partially over the top surface, wherein an upper surface of the insole liner coincides with the foot-contacting surface.

13. The insole of claim 12, wherein the insole liner has an overall shape and periphery matching an overall shape and periphery of the top surface of the insole support member.

14. The insole of claim 13, wherein the insole liner is an elastomer or plastic film, a mesh or a fabric.

15. The insole of claim 5, wherein the protective layer is a protective liner positioned over the top surface and at least partially covers the side-emitting optical fiber.

16. The insole of claim 15, wherein the protective liner has an overall shape and periphery matching an overall shape and periphery of the top surface of the insole support member.

17. The insole of claim 15, wherein the protective liner is made from light transmissive elastomer or plastic having a durometer of at least 50A.

18. The insole of claim 15, wherein at least one of the insole support member and the protective liner includes a channel, and the side-emitting optical fiber is received in the channel.

19. The insole of claim 15, wherein the protective liner includes a liner upper surface that coincides with the foot-contacting surface.

20. The insole of claim 1, further comprising an extension extending from a periphery of the insole support member, the extension configured to extend outwardly from an opening in the article of footwear configured to receive a wearer’s foot.

21. The insole of claim 20, wherein the side-emitting optical fiber is fixed to the extension.

22. An article of footwear comprising:
an upper;
a sole connected to the upper; and
the insole of claim 1.

23. The article of footwear of claim 22, further comprising a light pod including a light source, wherein the side-emitting optical fiber is optically connected to or optically connectable with the light source.

24. The article of footwear of claim 23, wherein the sole includes a light pod cavity and the light pod is received in the light pod cavity.

25. The article of footwear of claim 22, wherein the upper includes an opening for receiving a wearer’s foot and a passage extending from inside the upper to an exterior through which the side-emitting optical fiber passes.

26. The article of footwear of claim 25, further comprising a light pod including a light source, wherein the side-emitting optical fiber is optically connected to or optically connectable with the light source wherein the light pod is configured to attach to an outer surface of the upper.
